(19) United States
(12) Patent Application Publication
(10) Pub. No.: US 2012/0220884 A1
(43) Pub. Date: Aug. 30, 2012

(54) ELECTRONIC SPHYGMOMANOMETER, CONTROL METHOD FOR ELECTRONIC SPHYGMOMANOMETER, AND CONTROL PROGRAM FOR ELECTRONIC SPHYGMOMANOMETER

(75) Inventors: Shingo Yamashita, Kyoto (JP); Tomohiro Kukita, Amsterdam (NL)

(73) Assignee: OMRON HEALTHCARE Co., Ltd., Kyoto (JP)

(21) Appl. No.: 13/468,102

(22) Filed: May 10, 2012

Related U.S. Application Data
(63) Continuation of application No. PCT/JP2010/069712, filed on Nov. 5, 2010.

(30) Foreign Application Priority Data
Nov. 11, 2009 (JP) 2009-257865

(51) Int. Cl. A61B 5/022

(52) U.S. Cl. 600/490

(57) ABSTRACT

A sphygmomanometer has an automatic inflation mode and a manual inflation mode as modes for inflating a cuff. During control in the automatic inflation mode, the sphygmomanometer can determine whether the mode should be switched to the manual inflation mode. Upon switching to the manual inflation mode, when inflation is being carried out in the manual inflation mode, a mark indicating that inflation is being carried out in the manual inflation mode is displayed in a display region in a display screen.

### TECHNICAL FIELD

This invention relates to electronic sphygmomanometers, control methods for electronic sphygmomanometers, and control programs for electronic sphygmomanometers, and particularly relates to electronic sphygmomanometers, control methods for electronic sphygmomanometers, and control programs for electronic sphygmomanometers that measure blood pressures using a cuff that encloses an air bladder.

### BACKGROUND ART

Blood pressure is one index for analyzing cardiovascular disease, and performing a risk analysis based on blood pressure is effective in preventing cardiovascular-related conditions such as stroke, heart failure, and myocardial infarction. In particular, morning hypertension, in which the blood pressure rises in the early morning, is related to heart disease, stroke, and the like. Furthermore, among morning hypertension symptoms, the symptom called “morning surge”, in which the blood pressure rapidly rises within one hour to one and a half hours after waking up, has been found to have a causal relationship with stroke. Accordingly, understanding the interrelationship between time (lifestyle) and changes in blood pressure is useful in risk analysis for cardiovascular-related conditions. It is therefore necessary to continuously measure blood pressure over a long period of time.

Furthermore, recent study results have shown that blood pressure measured at home (home blood pressure) is more effective in the prevention, diagnosis, treatment, and so on of cardiovascular-related conditions than blood pressure measured at a hospital or during a health examination (casual blood pressure). Accordingly, sphygmomanometers for home use have become widely prevalent, and home blood pressure values have started to become used in diagnoses.

Typical sphygmomanometers are of a type in which during measurement, an arm band enclosing a cuff (a fluid bladder) is worn on a measurement area (that is, the upper arm or the like), compressing the measurement area until a blood vessel is closed off, after which the air is gradually released and the blood pressure is measured during the deflation of the cuff When measuring blood pressure using this type of sphygmomanometer, it is necessary to inflate the cuff until the blood vessel is closed off.

Some electronic sphygmomanometers, which measure blood pressures automatically, automatically determine the pressure to which to inflate the cuff. However, there are cases where proper inflation does not occur, due to arrhythmia, bodily movement, or the like. Accordingly, there are electronic sphygmomanometers provided with a function for determining inflation pressure based on additional operations performed by an operator. For example, JP H2-15105U discloses an automatic sphygmomanometer including an inflation bulb connected to an air flow channel, which includes both an automatic measurement mode, in which a cuff is automatically inflated/deflated, and a manual measurement mode, in which the cuff can be manually inflated/deflated using the inflation bulb.

### PATENT LITERATURE

Patent Literature 1: JP H2-15105U

### SUMMARY OF INVENTION

However, if the operator does not notice that the mode has switched from a control mode in which inflation occurs automatically to a control mode in which the inflation pressure is determined based on operations performed by the operator, the operator will not carry out the proper operations for determining the inflation pressure. Thus, the cuff may be improperly inflated, inflated too much, not inflated enough, or the like, which leads to unsuccessful blood pressure measurements.

Accordingly, one or more embodiments of the present invention provide an electronic sphygmomanometer, a control method for an electronic sphygmomanometer, and a control program for an electronic sphygmomanometer that can be used with ease by an operator.

According to one or more embodiments of the present invention, an electronic sphygmomanometer includes: an operation unit for accepting an operation; a fluid bladder; an adjustment unit for inflating and deflating the fluid bladder; a calculation unit for calculating a blood pressure value based on a change in an internal pressure of the fluid bladder; an informing unit; and a control unit for controlling the adjustment unit and information provided by the informing unit when the fluid bladder is inflated and deflated by the adjustment unit; here, the control unit controls the inflation performed by the adjustment unit selectively in one of a first control mode and a second control mode that serve as control modes in which the adjustment unit controls the inflation of the fluid bladder, and in at least one of the first control mode and the second control mode, causes the informing unit to provide information that the inflation is being controlled by the adjustment unit in the stated control mode. The control unit selects the first control mode as the control mode when a first operation has been detected on the operation unit, and selects the second control mode as the control mode when a second operation, which is an operation method different from the first operation, has been detected on the operation unit.

According to one or more embodiments of the present invention, the control unit determines that an operation for starting to operate has been made upon detecting that the first operation has been made on the operation unit. The first control mode is a control mode that, upon accepting an operation for starting to operate through the operation unit, automatically causes the adjustment unit to increase the internal pressure of the fluid bladder at a first inflation speed up to a predefined pressure; and the second control mode is a control mode that, upon accepting an operation through the operation unit, causes the adjustment unit to increase the internal pressure of the fluid bladder at a second inflation speed up to a specified pressure.

Here, the control unit has started the inflation of the fluid bladder in the first control mode due to the control unit detecting that the first operation has been made on the operation unit, and when the control unit has detected that the second operation has been made on the operation unit after the start of the inflation of the fluid bladder in the first control mode, according to one or more embodiments of the present invention, the control unit switches the control mode from the first control mode to the second control mode and continues, in the second control mode, the inflation of the fluid bladder that had been carried out in the first control mode.

According to one or more embodiments of the present invention, when the control unit is controlling the inflation performed by the adjustment unit in the second control mode, the control unit causes the informing unit to provide information to that effect.

Alternatively, according to one or more embodiments of the present invention, when the control unit is controlling the inflation performed by the adjustment unit in the second control mode, the control unit causes the informing unit to provide information to that effect and to make a notification that a stored pressure value is a recommended value.

According to one or more embodiments of the present invention, the second inflation speed to be lower than the first inflation speed.

Alternatively, according to one or more embodiments of the present invention, the second inflation speed is higher than the first inflation speed.

According to one or more embodiments of the present invention, when the calculation unit has returned an error in calculating the blood pressure value based on a change in the internal pressure of the fluid bladder whose inflation by the adjustment unit has been controlled in the first control mode, the control unit stores the second control mode as the next control mode.

According to one or more embodiments of the present invention, a control method for an electronic sphygmomanometer that has a fluid bladder, an operation unit, and a calculation unit for calculating a blood pressure value based on a change in an internal pressure of the fluid bladder includes: a step of starting the inflation of the fluid bladder by detecting a first operation made on the operation unit; a step of determining whether a control mode following the start of the inflation is to be set to a first control mode or a second control mode, in accordance with whether or not it has been detected that a second operation, which is an operation method different from the first operation, has been made on the operation unit after the inflation of the fluid bladder has started; a step of controlling the inflation of the fluid bladder according to a control method defined by the determined first control mode or second control mode; a step of communicating the determined control mode during the inflation of the fluid bladder; a step of ending the inflation and starting deflation when the internal pressure of the fluid bladder reaches a pressure defined by the determined control mode; a step of calculating a blood pressure value based on a change in the internal pressure of the fluid bladder during the deflation; and a step of outputting the blood pressure value.

According to one or more embodiments of the present invention, a program for causing an electronic sphygmomanometer that includes a fluid bladder, an operation unit, and a calculation unit for calculating a blood pressure value based on a change in an internal pressure of the fluid bladder to execute a blood pressure measurement operation includes: a step of starting the inflation of the fluid bladder by detecting a first operation made on the operation unit; a step of determining whether a control mode following the start of the inflation is to be set to a first control mode or a second control mode, in accordance with whether or not it has been detected that a second operation, which is an operation method different from the first operation, has been made on the operation unit after the inflation of the fluid bladder has started; a step of controlling the inflation of the fluid bladder according to a control method defined by the determined first control mode or second control mode; a step of communicating the determined control mode during the inflation of the fluid bladder; a step of ending the inflation and starting deflation when the internal pressure of the fluid bladder reaches a pressure defined by the determined control mode; a step of calculating a blood pressure value based on a change in the internal pressure of the fluid bladder during the deflation; and a step of outputting the blood pressure value.

The electronic sphygmomanometer according to one or more embodiments of the present invention can improve the ease of use for an operator.

### BRIEF DESCRIPTION OF DRAWINGS

FIG. 1 is a diagram illustrating a specific example of an external view of a sphygmomanometer according to one or more embodiments of the present invention.

FIG. 2 is a block diagram illustrating a specific example of the configuration of a sphygmomanometer according to one or more embodiments of the present invention.

FIG. 3 is a flowchart illustrating a specific example of operations performed by a sphygmomanometer according to one or more embodiments of the present invention.

FIG. 4 is a diagram illustrating a specific example of a display screen.

FIG. 5A is a diagram illustrating a specific example of a display screen.

FIG. 5B is a diagram illustrating a specific example of a display screen.

FIG. 5C is a diagram illustrating a specific example of a display screen.

### DETAILED DESCRIPTION OF INVENTION

Hereinafter, embodiments of the present invention will be described with reference to the drawings. In the following descriptions, identical reference numerals are added to identical components or constituent elements. The names and functions thereof are also the same.

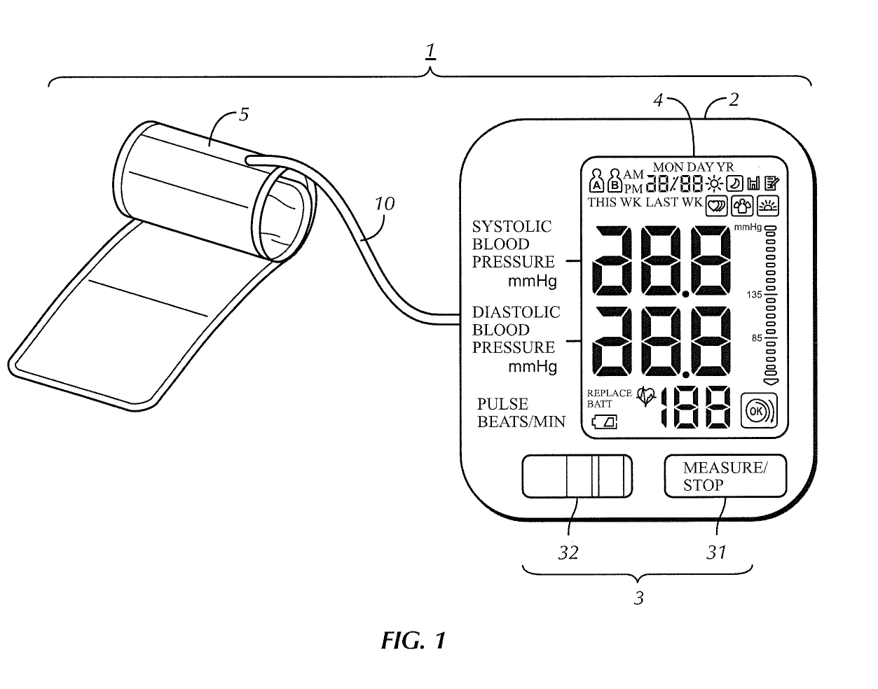

As shown in FIG. 1, an electronic sphygmomanometer (abbreviated to “sphygmomanometer” hereinafter) 1 according to one or more embodiments of the present invention includes a cuff 5, which serves as an air bladder, and a main body portion 2, and these constituent elements are connected by an air tube 10. A display unit 4 and an operation unit 3 are disposed on the front surface of the main body portion 2. The operation unit 3 includes a switch 31 that is used for operations such as turning the power on and off, starting and stopping measurement operations, and so on, and a switch 32 used in operations for selecting a measurement subject.

In measurements using the sphygmomanometer 1, when the measurement operation is started, the cuff 5 is inflated, thus pressurizing a measurement area on which the cuff 5 is worn such as the upper arm. After the cuff 5 has been inflated to a predetermined pressure at which the inner pressure of the cuff 5 is greater than or equal to the systolic blood pressure of the measurement subject, the inner pressure of the cuff 5 is gradually reduced; a blood pressure value of the measurement subject is calculated based on changes in the inner pressure of the cuff 5 during the deflation.

With the sphygmomanometer 1, two types of control are carried out selectively while the inner pressure of the cuff 5 is increased as mentioned above. One type is control that automatically inflates the cuff 5 to a predefined pressure at a predefined inflation speed, and the other type is control that, in accordance with an operation made through the switch 31, inflates the cuff 5 to a pressure specified through the operation at a predefined inflation speed. In the following descriptions, the former control mode in the inflation process will be called an “automatic inflation mode”, whereas the latter control mode in the inflation process will be called a “manual inflation mode”.

If the switch 31 of the sphygmomanometer 1 is depressed (is quickly pressed and released) when power is not being supplied to the sphygmomanometer 1, power is supplied to the sphygmomanometer 1 and the sphygmomanometer 1 enters a state in which measurement operations can be carried out. If the switch 31 is once again depressed (quickly pressed and released) in this state, the control mode of the sphygmomanometer 1 is set to the automatic inflation mode, and inflation of the cuff 5 begins. During the inflation process, when the switch 31 of the sphygmomanometer 1 is depressed continuously for greater than or equal to a predetermined amount of time (is held down), the control mode for the inflation process switches from the automatic inflation mode to the manual inflation mode, and the inner pressure of the cuff 5 is increased at an inflation speed according to the manual inflation mode while the switch 31 is being held down. When the switch 31 is released, the inflation of the cuff 5 ends, and the deflation process then commences. After this, when the measurement operation ends, the sphygmomanometer 1 automatically turns off.

In the case where, during inflation in the automatic inflation mode, the operator has inadvertently held down the switch 31, the deflation process will commence after the switch 31 has been released, and the power will be turned off when the measurement operation ends thereafter. At this time, if the operator has not realized that the manual inflation mode is in effect, the deflation process will begin unintentionally, and the power will be turned off thereafter; in this case, the series of operations will end with insufficient inflation, and thus without a proper blood pressure value having been calculated. In other words, the usability will be extremely poor unless the operator can confirm that the control mode during inflation has switched to the manual inflation mode. Accordingly, the sphygmomanometer 1 includes a function for communicating at least that the control mode during inflation has switched to the manual inflation mode when such a switch has occurred.

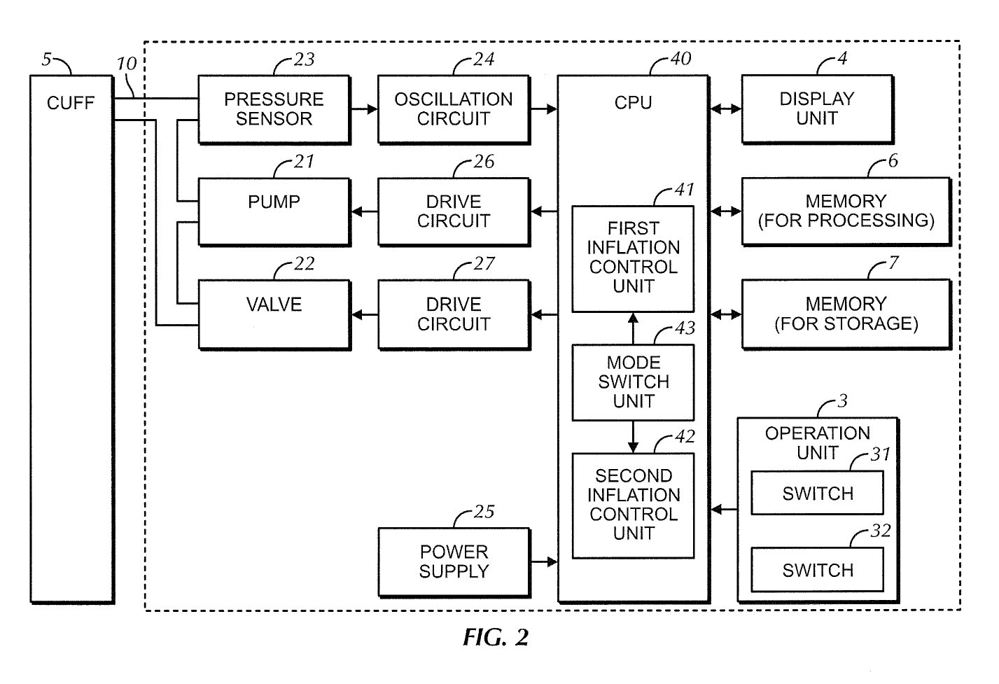

As shown in FIG. 2, the main body portion 2 of the sphygmomanometer 1 includes a pressure sensor 23, a pump 21, and a valve 22 connected to the cuff 5 by the air tube 10. The pressure sensor 23 is connected to an oscillation circuit 24. The pump 21 is connected to a drive circuit 26, and the valve 22 is connected to a drive circuit 27.

The oscillation circuit 24, the drive circuit 26, and the drive circuit 27 are connected to a CPU (Central Processing Unit) 40 that controls the sphygmomanometer 1 as a whole. Furthermore, the display unit 4, the operation unit 3, a memory 6 for processing, a memory 7 for recording, and a power supply 25 are connected to the CPU 40. The memory 6 for processing stores control programs and so on executed by the CPU 40. The memory 6 for processing also serves as a work area used when the CPU 40 executes programs.

The CPU 40 is driven having received a supply of power from the power supply 25. The CPU 40 executes predetermined programs stored in the memory 6 based on operation signals inputted from the operation unit 3, and outputs control signals to the drive circuit 26 and the drive circuit 27. The drive circuit 26 and the drive circuit 27 drive the pump 21 and the valve 22 based on the control signals. The driving of the pump 21 is controlled by the drive circuit 26 in accordance with the control signals from the CPU 40, and air is injected into the cuff 5 as a result. The opening/closing of the valve 22, meanwhile, is controlled by the drive circuit 27 in accordance with the control signals from the CPU 40, and air is released from the cuff 5 as a result.

The pressure sensor 23 is an electrostatic capacitance-type pressure sensor, and a capacity value thereof changes as the interior pressure of the cuff 5 changes. The oscillation circuit 24 inputs a signal having an oscillation frequency based on the capacity value of the pressure sensor 23 to the CPU 40. The CPU 40 executes a predetermined process based on the change in the interior pressure of the cuff 5 obtained from the pressure sensor 23, and outputs the stated control signals to the drive circuit 26 and the drive circuit 27 in accordance with the result of the process. In addition, the CPU 40 outputs a blood pressure value based on the change in the interior pressure of the cuff 5 obtained from the pressure sensor 23, carries out a process for displaying a result of the measurement in the display unit 4, and outputs data and a control signal for carrying out the display to the display unit 4. Furthermore, the CPU 40 carries out a process for storing the blood pressure value in the memory 7.

The CPU 40 includes a first inflation control unit 41, a second inflation control unit 42, and a mode switch unit 43. These are primarily functions implemented by the CPU 40, by the CPU 40 reading out and executing the stated control programs stored in the memory 6 in accordance with operation signals from the operation unit 3, but at least some of these functions may be implemented through a hardware configuration as indicated in FIG. 1.

The first inflation control unit 41 and the second inflation control unit 42 are functions for control carried out during the stated inflation process of the cuff 5; the first inflation control unit 41 is a function for control carried out in the stated automatic inflation mode, whereas the second inflation control unit 42 is a function for control carried out in the manual inflation mode. The mode switch unit 43, meanwhile, stores the current control mode, determines the control mode during the inflation process based on an operation signal from the switch 31 and the current control mode, and switches the current control mode as necessary. In other words, a signal is outputted to the first inflation control unit 41 when inflation starts in the automatic inflation mode, thus starting control in the automatic inflation mode. When, during control in the automatic inflation mode, it is detected that the switch 31 is being held down based on the operation signal inputted from the switch 31, it is determined that the mode is to be switched to the manual inflation mode; a signal is then outputted to the second inflation control unit 42, and control starts in the manual inflation mode. Along with this, the mode switch unit 43 outputs a signal to the display unit 4, thus causing the display unit 4 to display that the mode is the manual inflation mode. When the first inflation control unit 41 and the second inflation control unit 42 receive the stated signals from the mode switch unit 43, the necessary control programs are read out from the memory 6 and executed.

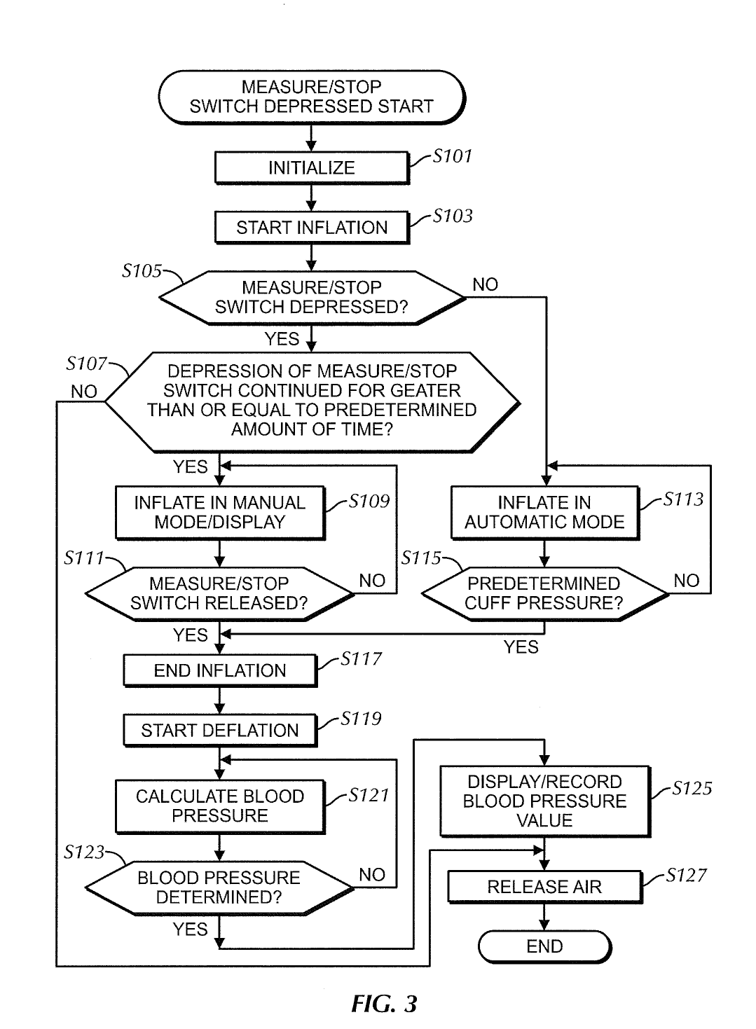

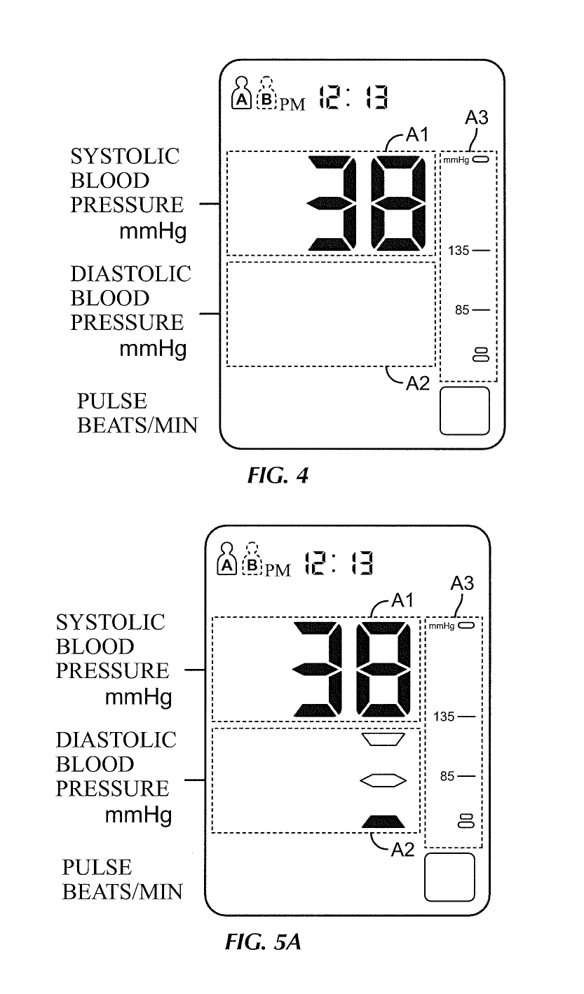

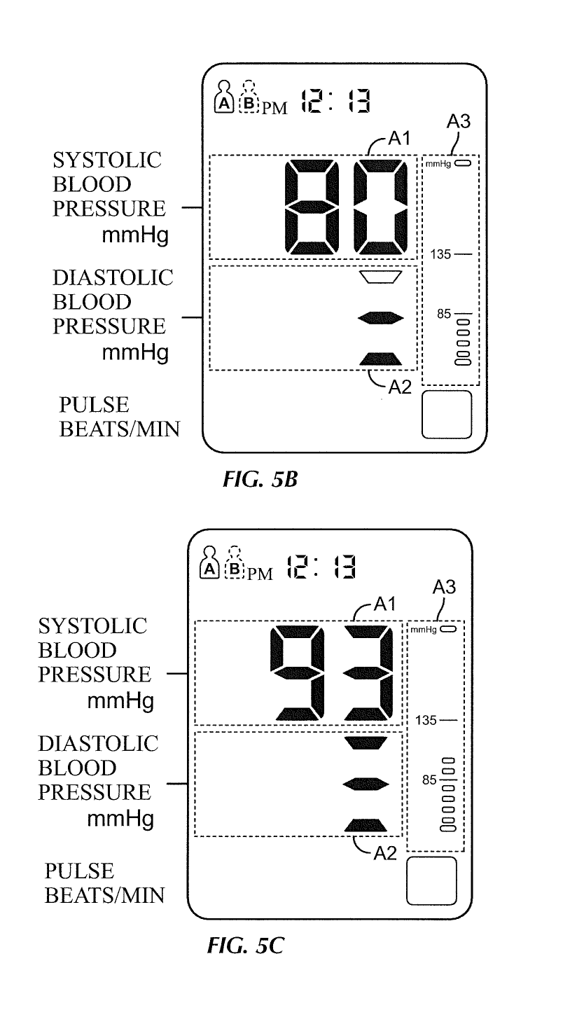

FIG. 3, FIG. 4, and FIG. 5A through FIG. 5C illustrate operations and display screens of the sphygmomanometer 1. The operations illustrated in the flowchart shown in FIG. 3 are realized when the switch 31 is depressed, power is supplied to the CPU 40 from the power supply 25, the control programs stored in the memory 6 for processing are read out, and the various elements illustrated in FIG. 1 and FIG. 2 are controlled.

Referring to FIG. 3, when operations are started, the CPU 40 initializes the various elements in step S101, and starts the inflation of the cuff 5 in step S103. At this time, the mode switch unit 43 determines that the control mode during the inflation process is the automatic inflation mode, which serves as a default mode, and outputs a signal to the first inflation control unit 41 to cause the first inflation control unit 41 to operate. Due to the first inflation control unit 41 controlling the inflation operation in accordance with the signal, a control signal is outputted from the CPU 40 to the drive circuit 26, and the cuff 5 is inflated at a predefined, predetermined inflation speed.

Furthermore, in accordance with the first inflation control unit 41 executing the control program, the CPU 40 outputs, to the display unit 4, a signal indicating the internal pressure of the cuff 5 that has been inputted through the oscillation circuit 24 and a control signal for display. Through this, a screen such as that shown in FIG. 4 is displayed in the display unit 4 during the inflation process in the automatic inflation mode. In other words, as shown in FIG. 4, when displaying a result of the measurement in the display screen, the current internal pressure of the cuff 5 is displayed in a region A1 that displays a systolic blood pressure value, and nothing is displayed in a region A2 that displays a diastolic blood pressure value; changes in the internal pressure of the cuff 5 are displayed as segments in a region A3, which is next to the regions A1 and A2 and in which scale marks are provided. According to one or more embodiments of the present invention, the internal pressure of the cuff 5 is displayed in the regions A1 and A2 using digital segments, which are used to display blood pressure values. Doing so makes a new display region unnecessary, which in turn suppresses an increase in the size of the sphygmomanometer 1.

When it is detected during the inflation of the cuff 5 that the switch 31 has been depressed and it has also been detected that the switch 31 has been held down, or depressed for greater than or equal to a predefined amount of time (YES in S105 and YES in S107), in step S109, the mode switch unit 43 determines to switch the control mode in the inflation process from the current automatic inflation mode to the manual inflation mode, and outputs a signal for operating the second inflation control unit 42 to the second inflation control unit 42. Along with this, the mode switch unit 43 outputs, to the display unit 4, a signal for carrying out a display of the automatic inflation mode. Due to the second inflation control unit 42 controlling the inflation operation in accordance with the signal, a control signal is outputted from the CPU 40 to the drive circuit 26, and the cuff 5 is inflated at a predefined, predetermined inflation speed.

Furthermore, in accordance with the second inflation control unit 42 executing the control program, the CPU 40 outputs, to the display unit 4, a signal indicating the internal pressure of the cuff 5 that has been inputted through the oscillation circuit 24 and a control signal for display. Through this, a screen such as that shown in FIG. 5A through FIG. 5C is displayed in the display unit 4 during the inflation process in the manual inflation mode. In other words, as shown in FIG. 5A, the current internal pressure of the cuff 5 is displayed in the region A1 of the display screen in the same manner as when in the automatic inflation mode. Along with this, a mark indicating that the current control mode is the manual inflation mode is displayed in the region A2 of the display screen based on the signal from the mode switch unit 43. In the example shown in FIG. 5A, the manual inflation mode is indicated by a mark that appears when some of the digital segments used to display the diastolic blood pressure value in the region A2 are lit up.

In accordance with the execution of the control program by the second inflation control unit 42, the CPU 40 monitors the operation signal inputted from the switch 31, and detects when this input has ended, or in other words, detects when the switch 31 has been released. When the CPU 40 has detected that the switch 31 has been released (YES in step S111), in step 5117, the second inflation control unit 42 ends the inflation control, and in step S119, the CPU 40 starts deflating the cuff 5. Until the switch 31 is released, or in other words, while the switch 31 is being held down (NO in step S111), the inflation of the cuff 5 in step S109 is continued.

While the inflation continues in the manual inflation mode, the display in the display unit 4 is carried out as shown in FIG. 5A, FIG. 5B, and FIG. 5C, where the internal pressure of the cuff 5 increases as indicated in the region A1 and the number of lit segments that express a change in the internal pressure increases as indicated in the region A3; furthermore, according to one or more embodiments of the present invention, the mark shown in the region A2 that indicates the manual inflation mode changes to express the increase in pressure. Specifically, the mark that indicates the manual inflation mode employs the three horizontal lines in the digital segments used in the display of the diastolic blood pressure value. During inflation, the inflation of the cuff 5 is expressed by the lit horizontal lines increasing from the bottom up, and when all horizontal lines have become lit, returning to the bottom line being lit and once again increasing the lit horizontal lines from the bottom up.

While confirming the current internal pressure of the cuff 5 displayed in the region A1 in FIG. 5A through FIG. 5C, the operator releases the switch 31 when the desired pressure has been reached, thus specifying that the cuff 5 has been inflated to that pressure. According to one or more embodiments of the present invention, the inflation speed in the manual inflation mode is set in advance to be lower than the inflation speed in the automatic inflation mode. By making settings in this manner, the operator can ensure that the desired pressure is reached with certainty, which makes it possible to increase the internal pressure of the cuff 5 to the desired pressure.

Alternatively, the inflation speed in the manual inflation mode may be set in advance to be higher than the inflation speed in the automatic inflation mode. By making such a setting, the inflation of the cuff 5 can be ended more quickly, which makes it possible to reduce the amount of time required for the overall measurement operation. Furthermore, in the case where the inflation speed in the manual inflation mode is set to be higher than the inflation speed in the automatic inflation mode, according to one or more embodiments of the present invention, the inflation speed is provided with two stages, so that the inflation speed decreases as the internal pressure of the cuff 5 approaches a predetermined pressure. The stated predetermined pressure may, for example, be a pressure stored in advance as a pressure that is near the typical maximum inflation point, may be a pressure near the pressure obtained and stored the previous time the cuff 5 was inflated when measuring the measurement subject in question, or may be a pressure near a pressure in which a predetermined pressure (for example, 40 mmHg) has been added to a value obtained by estimating the systolic blood pressure value of the measurement subject during the inflation process. Doing so makes it possible for the operator to confirm with certainty that the desired pressure has been reached, which in turn makes it possible to accelerate the inflation of the cuff 5 while increasing the internal pressure of the cuff 5 to the desired pressure.

Furthermore, in addition to displaying an indication that the control mode is the manual inflation mode in the display unit 4 during inflation in the manual inflation mode, according to one or more embodiments of the present invention, the CPU 40 displays a pre-stored pressure as a recommended pressure value. The recommended pressure value may, for example, use a pressure value obtained and stored the previous time the cuff 5 was inflated when measuring the measurement subject in question, may be a pressure value in which a predetermined pressure (for example, 40 mmHg) has been added to a value obtained by estimating the systolic blood pressure value of the measurement subject during the inflation process, or may be a pressure value stored in advance as a pressure that is near a typical maximum inflation point.

In the case where a depression of the switch 31 has not been detected after the cuff 5 has started being inflated in the automatic inflation mode in step S103 (NO in step S105), inflation control is continued in the automatic inflation mode by the first inflation control unit 41 until the internal pressure of the cuff 5 has reached a predefined, predetermined pressure (step S113). When the internal pressure of the cuff 5 has reached the predetermined pressure (YES in step S115), in step S117, the first inflation control unit 41 ends the inflation control, and in step S119, the CPU 40 starts deflating the cuff 5.

When the cuff 5 begins deflating in step S119, in step S121, the CPU 40 extracts a vibration component resulting from a change in the volume of the artery superimposed on the internal pressure of the cuff 5 obtained during the deflation, and calculates a blood pressure value through a predetermined process. When the blood pressure value has been determined in the calculation process of step S121 (YES in step S123), in step S125, the CPU 40 carries out a process for displaying the calculated systolic blood pressure value and diastolic blood pressure value in the display unit 4 as measurement results. Furthermore, in step S127, the CPU 40 outputs control signals to the drive circuits 26 and 27, and the pressure within the cuff 5 is restored to the atmospheric pressure.

According to one or more embodiments of the present invention, the CPU 40 monitors whether or not the blood pressure value has been determined within a predetermined amount of time, and in the case where the blood pressure value has not been determined within the predetermined amount of time due to insufficient internal pressure in the cuff 5 or the like, a measurement error may be returned. In this case, the mode switch unit 43 may store the manual inflation mode in, for example, a region for storing the current operation mode, as the inflation control mode to be used for the measurement subject in question the next time measurement is carried out. Such being the case, the mode switch unit 43 may determine the manual inflation mode to be used as the default inflation control mode when measurement operations are carried out for that measurement subject, or may carry out a display, in the display unit 4, prompting the measurement to be carried out in the manual inflation mode, which corresponds to a recommended control mode.

It should be noted that if, after the cuff 5 has started being inflated in the automatic inflation mode in step S103 as mentioned above, it is both detected that the switch 31 has been depressed and it is detected that the switch 31 has been quickly pressed and released, in which the depression is shorter than a predefined amount of time (YES in S105 and NO in S107), the CPU 40 determines that the measurement operations are to be ended, and in step S127, outputs control signals to the drive circuits 26 and 27 and restores the pressure within the cuff 5 to the atmospheric pressure.

The series of operations ends when the pressure within the cuff 5 is restored to the atmospheric pressure; after this, the supply of power from the power supply 25 is automatically interrupted, thus turning the power off.

Although the aforementioned example describes the manual inflation mode as the only inflation control mode that is indicated using a display, it should be noted that both modes may be indicated using a display. Furthermore, although a specific example is described in which some of the digital segments are lit up in order to display the blood pressure value, the control mode may be communicated using a different method than a display carried out in the display unit 4; for example, a display region for presenting the inflation control mode may further be provided, the display state may be changed by, for example, changing the background color of the display screen, a lamp may be provided, audio may be outputted, or the like.

In this manner, according to the sphygmomanometer 1, when the manual inflation mode is selectively executed as the control mode for inflating the cuff 5, a notification to that effect is made. As a result, it is possible for the operator to understand whether the current inflation control mode is the manual inflation mode or the automatic inflation mode.

Meanwhile, there are situations where, in order to reduce the size of the sphygmomanometer, the design is such that the number of operational switches is reduced and a single switch is used to carry out multiple operations. In the case where the design is such that both an operation specifying the start of measurement and an operation for switching from the automatic inflation mode to the manual inflation mode can be carried out using a single switch, as illustrated in FIG. 1, it is particularly easy for the operator to inadvertently switch from the automatic inflation mode to the manual inflation mode as was described above. According to the sphygmomanometer 1, however, even in such a case, the operator can visually confirm the current control mode from the display unit 4, and can therefore properly specify the pressure to be used when inflating the cuff 5, which makes it possible to continue the measurement operations. In other words, communicating the current inflation control mode makes it possible to improve the ease of use for the operator, particularly in a sphygmomanometer in which a switch for switching the inflation control mode is not provided and a function for doing so is implemented through a switch used for other operations as well.

Furthermore, a program for causing the sphygmomanometer 1 to execute the aforementioned operations can also be provided. Such a program can be recorded on a computer-readable recording medium such as a flexible disk, a CD-ROM (compact disc read-only memory), a ROM (read-only memory), a RAM (random access memory), or a memory card that is supplied to a computer, and can be provided in such form as a program product. Alternatively, the program can be recorded on a recording medium such as a hard disk mounted within a computer, and can be provided in such form as a program. Further still, the program can also be downloaded via a network, and can be provided in such form as a program.

Note that the program according to one or more embodiments of the present invention may execute processing by calling, in a predetermined arrangement and at a predetermined timing, the necessary program modules from among the modules provided as part of an operating system (OS) of a computer. In this case, the stated modules are not included in the program itself, and the processing is executed in cooperation with the OS. Such a program that does not include modules in this manner can also fall within the scope of the program according to one or more embodiments of the present invention.

In addition, the program according to one or more embodiments of the present invention may be provided having been incorporated into a part of another program. In such a case as well, modules included in the stated other program are not included within the program itself, and the processing is executed in cooperation with the other program. Such a program that is incorporated into another program can also fall within the scope of the program according to one or more embodiments of the present invention.

The program product that is provided is installed in a program storage unit such as a hard disk and executed. Note that the program product includes the program itself and the recording medium on which the program is recorded.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCE NUMERALS

1 sphygmomanometer

2 main body portion

3 operation unit

4 display unit

5 cuff

6, 7 memory

10 air tube

21 pump

22 valve

23 pressure sensor

24 oscillation circuit

25 power supply

26, 27 drive circuit

31, 32 switch

40 CPU

41 first inflation control unit

42 second inflation control unit

43 mode switch unit

1. An electronic sphygmomanometer comprising:
an operation unit that accepts an operation;
a fluid bladder;
an adjustment unit that inflates and deflates the fluid bladder;
a calculation unit that calculates a blood pressure value based on a change in an internal pressure of the fluid bladder;
an informing unit; and
a control unit that controls the adjustment unit and information provided by the informing unit when the fluid bladder is inflated and deflated by the adjustment unit,
wherein the control unit controls the inflation performed by the adjustment unit selectively in one of a first control mode and a second control mode that serve as control modes in which the adjustment unit controls the inflation of the fluid bladder, and in at least one of the first control mode and the second control mode, causes the informing unit to provide information that the inflation is being controlled by the adjustment unit in the stated control mode, and
wherein the control unit selects the first control mode as the control mode when a first operation has been detected on the operation unit, and selects the second control mode as the control mode when a second operation, which is an operation method different from the first operation, has been detected on the operation unit.

2. The electronic sphygmomanometer according to claim 1,
wherein the control unit determines that an operation for starting to operate has been made upon detecting that the first operation has been made on the operation unit,
wherein the first control mode is a control mode that, upon accepting the operation for starting to operate through the operation unit, automatically causes the adjustment unit to increase the internal pressure of the fluid bladder at a first inflation speed up to a predefined pressure, and
wherein the second control mode is a control mode that, in response to the second operation accepted through the operation unit, causes the adjustment unit to increase the internal pressure of the fluid bladder at a second inflation speed up to a specified pressure.

3. The electronic sphygmomanometer according to claim 1,
wherein the control unit has started the inflation of the fluid bladder in the first control mode due to the control unit detecting that the first operation has been made on the operation unit, and when the control unit has detected that the second operation has been made on the operation unit after the start of the inflation of the fluid bladder in the first control mode, the control unit switches the control mode from the first control mode to the second control mode and continues, in the second control mode, the inflation of the fluid bladder that had been carried out in the first control mode.

4. The electronic sphygmomanometer according to claim 2,
wherein when the control unit is controlling the inflation performed by the adjustment unit in the second control mode, the control unit causes the informing unit to provide information to that effect.

5. The electronic sphygmomanometer according to claim 2,
wherein when the control unit is controlling the inflation performed by the adjustment unit in the second control mode, the control unit causes the informing unit to provide information to that effect and to make a notification that a stored pressure value is a recommended value.

6. The electronic sphygmomanometer according to claim 2,
wherein the second inflation speed is lower than the first inflation speed.

7. The electronic sphygmomanometer according to claim 2,
wherein the second inflation speed is higher than the first inflation speed.

8. The electronic sphygmomanometer according to claim 2,
wherein when the calculation unit has returned an error in calculating the blood pressure value based on a change in the internal pressure of the fluid bladder whose inflation by the adjustment unit has been controlled in the first control mode, the control unit stores the second control mode as the next control mode.

9. A control method for an electronic sphygmomanometer that comprises a fluid bladder, an operation unit, and a calculation unit that calculates a blood pressure value based on a change in an internal pressure of the fluid bladder, the method comprising:
a step of starting an inflation of the fluid bladder by detecting a first operation made on the operation unit;
a step of determining whether a control mode following the start of the inflation is to be set to a first control mode or a second control mode, in accordance with whether or not it has been detected that a second operation, which is an operation method different from the first operation, has been made on the operation unit after the inflation of the fluid bladder has started;
a step of controlling the inflation of the fluid bladder according to a control method defined by the determined first control mode or second control mode;
a step of communicating the determined control mode during the inflation of the fluid bladder;
a step of ending the inflation and starting deflation when the internal pressure of the fluid bladder reaches a pressure defined by the determined control mode;
a step of calculating a blood pressure value based on a change in the internal pressure of the fluid bladder during the deflation; and
a step of outputting the blood pressure value.

10. A program that causes an electronic sphygmomanometer that comprises a fluid bladder, an operation unit, and a calculation unit that calculates a blood pressure value based on a change in an internal pressure of the fluid bladder to execute a blood pressure measurement operation comprising:
a step of starting an inflation of the fluid bladder by detecting a first operation made on the operation unit;
a step of determining whether a control mode following the start of the inflation is to be set to a first control mode or a second control mode, in accordance with whether or not it has been detected that a second operation, which is an operation method different from the first operation, has been made on the operation unit after the inflation of the fluid bladder has started;
a step of controlling the inflation of the fluid bladder according to a control method defined by the determined first control mode or second control mode;
a step of communicating the determined control mode during the inflation of the fluid bladder;
a step of ending the inflation and starting deflation when the internal pressure of the fluid bladder reaches a pressure defined by the determined control mode;
a step of calculating a blood pressure value based on a change in the internal pressure of the fluid bladder during the deflation; and
a step of outputting the blood pressure value.

11. The electronic sphygmomanometer according to claim 2,
wherein the control unit has started the inflation of the fluid bladder in the first control mode due to the control unit detecting that the first operation has been made on the operation unit, and when the control unit has detected that the second operation has been made on the operation unit after the start of the inflation of the fluid bladder in the first control mode, the control unit switches the control mode from the first control mode to the second control mode and continues, in the second control mode, the inflation of the fluid bladder that had been carried out in the first control mode.
